(19) United States
(12) Plant Patent Application Publication
(10) Pub. No.: US 2003/0217398 P1
(43) Pub. Date: Nov. 20, 2003

(54) Hybrid tea rose plant named ' RUIY5451'

(76) Inventor: H.C.A. de Groot, De Kwakel (NL)

Correspondence Address: CHRISTIE, PARKER & HALE, LLP, 350 WEST COLORADO BOULEVARD, SUITE 500, PASADENA, CA, 91105, US

(21) Appl. No.: 10/406,918

(22) Filed: Apr. 4, 2003

(30) Foreign Application Priority Data
May 14, 2002 (EP) 2002/0749
Sep. 10, 2002 (PL) O1349

(51) Int. Cl. A01H 5/00

(52) U.S. Cl. PLT/137

(57) ABSTRACT

A new and distinct hybrid tea rose plant having attractive large flowers.

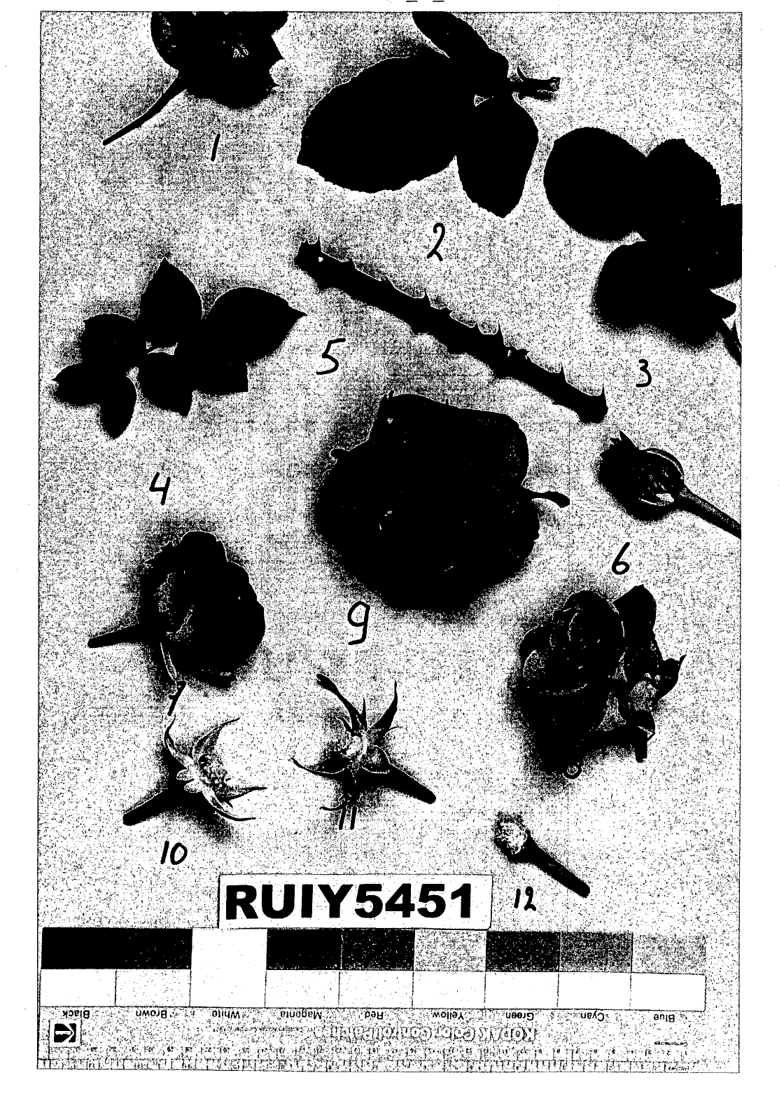

### BACKGROUND OF THE INVENTION

The present invention relates to a new and distinct variety of rose plant of the hybrid tea rose class, which was created by crossing an undistributed, unpatented seedling as seed parent with the variety known as ‘PANNARAN’ as pollen parent. The varietal denomination of this new rose is named ‘RUIY5451’.

### SUMMARY OF THE INVENTION

Among the novel characteristics possessed by this new variety which distinguishes it from its parent and all other varieties of which I am aware are its flower color.

Asexual reproduction by propagation by grafting and by vegetative cuttings of the new variety as performed in De Kwakel, The Netherlands shows that the foregoing and other distinguishing characteristics come true to form and are established and transmitted through succeeding propagation.

### COMPARISON WITH PARENT

The new variety differs from its parents in flower color, inasmuch as the flowers of ‘RUIY5451’ are orange red and flowers of the seed parent are cream and light pink and flowers of the pollen parent are salmon.

### BRIEF DESCRIPTION OF THE ILLUSTRATION

The accompanying illustration shows a typical specimen of the new variety depicted in color as nearly true as is reasonably possible to make the same in color illustrations of this character.

In the illustration, 1, 2, 3 and 4 show the foliage; 5 shows a stem portion; 6, 7, 8 and 9 show a bud and blooms at different stages of development; and 10, 11 and 12 show the reproductive organs.

### DESCRIPTION OF THE NEW VARIETY

The following is a detailed description of the new variety with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of 9-month old plants grown in a greenhouse in De Kwakel, The Netherlands during January.

### FLOWER

Blooming habit: Continuous.

Bud:

Size.—Large, about 5-6 cm. long by about 4 cm. wide.

Form.—Ovate.

Color.—When sepals first divide, orange near 33A.

Sepals.—Just before opening sepals are as long as the bud; color top surface near 146C to 146D; under surface near 146C to 146D and sometimes in the middle a small part near 46B.

Peduncle:

Length.—About 11 cm.

Aspect.—Smooth.

Strength.—Erect, strong.

Color.—Near 138A.

### BLOOM

Size: Average size when fully open is 14 cm.

Borne: Singly.

Form: High centered at first; becoming flat; petals remaining at first loosely rolled outward, becoming ruffled at maturity.

Petalage:

Number of petals under normal conditions.—About 25.

Color (⅓ to ½ open):

Outer petals.—Top surface: Near 40B. Under Surface: Near 42B.

Inner petals.—Top Surface: Near 40B. Under Surface: Near 42B to 42C.

Base of petals (Point of attachment).—Near 13A.

General tonality from a distance.—Orange pink.

Color (Fully open):

Outer petals.—Top Surface: Near 40B. Undersurface: Near 42B.

Inner petals.—Top Surface: Near 40B. Undersurface: Near 42B.

Base of petals (Point of attachment).—Near 13A.

General tonality from a distance.—Orange pink.

Color change:

As bloom ages.—Petal Top Surface: Near 63B. Petal Under Surface: Near 63C to 63D. General tonality from a distance: Petal color becomes dull pink.

### PETALS

Texture: Smooth.

Appearance: Inside and outside satiny; reflexing of margin is weak.

Form: Shell-shaped.

Arrangement: Regularly arranged, imbricated.

Petaloids in center: Few, about 4; same color as petals.

Persistence: Petals drop off cleanly.

Fragrance: No fragrance.

Lasting quality on plant after cutting: On the plant about 18 days; as cut flower, vase life about 11 days.

### REPRODUCTIVE ORGANS

Arrangement: Stamens, regularly arranged around styles.

Color:

Filaments.—Yellow orange.

Anthers.—Near 20A and 22A.

Pollen:

Color.—Brownish yellow.

Styles: Uneven length about 1 cm.

Stigmas:

Color.—Cream about near 11B.

Hips:

Shape.—Pitcher shaped.

Color (when mature).—Pale orange green.

Seeds.—Some seeds protrude.

### PLANT

Form: Upright.

Growth: Vigorous, uniform branching.

Height attained (first season).—About 200 cm.

Foliage: Compound 3, 5 and 7 leaflet leaves.

Size: Largest leaflets — length about 12-8 cm., width about 8-5 cm.; shoot length about 8 cm.

Quantity: Usually about 14 leafs per stem.

Color:

New foliage.—Upper Side: Purple to bronze, near 183B. Under Side: Near 178A.

Old foliage.—Upper Side: Near 139A. Under Side: Near 137C.

Shape: Elliptic.

Texture:

Upper side.—Weak to medium glossy.

Under side.—Matte, veins are clearly visible.

Edge: Serrated.

Serration type: Ordinary, accumunate pieces.

Petiole:

Color.—Top Surface: In center near 138B; sides near 183B. Under Side: Near 138B.

Stipules: Slightly bearded.

Length.—0.5 cm.

Width.—About 1 cm.

Color.—About 138B to 138C.

Auricle:

Length.—Up to about 2 cm.

Width.—About 0.8 cm.

Color.—138B to near 183B.

Rachis:

Surface.—Top is flat.

Under side.—Rounded, with some prickles.

Resistance to disease: Susceptible to blackspot, mildew, and rust.

### WOOD

New shoots:

Stem.—About 10 cm.; green near 138B.

Bark.—Rough, with many small prickles.

Old wood:

Color.—Green.

Bark.—Smooth, green with thorns near 138A.

Flower stem:

Length from last cut (including peduncle).—Average about 80 cm.

Thorns: About 6 thorns per internode.

On laterals from basal canes.—Many.

Form.—Curved downwardly.

Length.—Medium, about 1 cm.

Color (when young).—Brownish purple near 187B.

Color (when old).—Brownish green near 177B.

Position.—Irregular.

Prickles: Normal; same color as thorns.

Winter hardiness: Not known.

Uniqueness: Very large bud size, flower color; good production, about 200 stems/m²/year.

I claim:

1. A new and distinct hybrid tea rose plant of the variety substantially as shown and described.
